(19) United States
(12) Patent Application Publication
(10) Pub. No.: US 2012/0128633 A1
(43) Pub. Date: May 24, 2012

(54) USE OF COLLINSELLA AEROFACIENS FOR REDUCING BLOATING

(75) Inventors: Patrick Veiga, Suresnes (FR); Raish Oozber, Montgeron (FR); Karine Roy, Palaiseau (FR); Stephanie Cools-Portier, Epinay sur Orge (FR); Aurore Tessier, Laquueue les Yvelines (FR); Pascale Rondeau, Savigny sur Orge (FR); Denis Guyonnet, Levallois-Perret (FR)

(73) Assignee: CAMPAGNIE GERVAISE DONONE, Paris (FR)

(21) Appl. No.: 13/318,133

(22) PCT Filed: Apr. 30, 2009
(86) PCT No.: PCT/IB09/05735

§ 371 Date: Feb. 10, 2012

(51) Int. Cl. A61K 35/74; A61P 1/00

(52) U.S. Cl. 424/93.4

(57) ABSTRACT

The invention relates to compositions comprising Collinsella aerofaciens for use for reducing bloating, in particular in subjects suffering from irritable bowel syndrome.

The invention relates to the treatment of bowel disorders, and more particularly of irritable bowel syndrome.

Irritable bowel syndrome (IBS) is a common bowel disorder, affecting up to 15% of the Western population. It is characterised by a mixture of symptoms, including abdominal pain or discomfort, bloating, and constipation, diarrhea, or both. IBS has been sub-classified into IBS with constipation (IBS-C), IBS with diarrhea (IBS-D), or IBS with alternating constipation or diarrhea symptoms (IBS-M).

Although several causes, including stress, food intolerances, and an imbalance of the intestinal microflora have been identified, the etiology of IBS is poorly understood. Therefore, at the moment, there is no global cure for this disease, and the treatment is focused on relieving symptoms.

Bloating is an extremely common symptom of irritable bowel syndrome, and is one of the most bothersome.

There is growing evidence that IBS is associated with alterations in the gastro-intestinal microflora (Malinen et al., Am J Gastroenterol, 100, 373-82, 2005; Matto et al., FEMS Immunol Med Microbiol, 43, 213-22, 2005; Maukonen et al., J Med Microbiol, 55, 625-33, 2006;. Kassinen et al., Gastroenterology,133, 24-33, 2007), and ingestion of probiotic bacteria has been reported to result in alleviation of some of the symptoms of IBS (for review cf. for instance Parkes et al., Am J Gastroenterol, 103, 1557-67, 2008).

For instance it has been shown that a fermented dairy product containing the Bifidobacterium lactis strain DN-173 010 improved the symptoms of bloating and digestive discomfort, and reduced abdominal distension in patients with IBS-C (Guyonnet et al., Aliment Pharmacol Ther, 26, 475-86, 2007; Agrawal et al., Aliment Pharmacol Ther, 29, 104-114, 2008).

The inventors have now found that the effect of Bifidobacterium lactis DN-173 010 on reduction of bloating is closely correlated with an increase in the intestinal population of Collinsella aerofaciens in the treated subjects.

This finding of the inventors allows to propose the use of Collinsella aerofaciens for treating or preventing bloating and/or the feeling of bloating.

Collinsella aerofaciens (formerly Eubacterium aerofaciens; Moore et al., Int J Syst Bacteriol, 21, 307-310, 1971; Kageyama et al., Int J Syst Bacteriol, 49, 557-565, 1999) belongs to the class of Actinobacteria.

An object of the present invention is therefore a composition comprising Collinsella aerofaciens for use for reducing intestinal bloating and/or the feeling of bloating, preferably for use in a subject suffering from irritable bowel syndrome, in particular from IBS-C.

Strains of Collinsella aerofaciens which are more particularly suitable for use in the present invention are non arthritogenic strains, which can easily be identified for instance on the basis of the sensitivity of their cell walls to enzymatic digestion with lysozyme or with mutanolysin (Zhang et al., Infect. Immun., 69, 7277-7284, 2001).

In a preferred embodiment of the invention, said composition comprises from about 10³ CFU/ml to about 10¹¹ CFU/ml of Collinsella aerofaciens. It may also comprise other bacteria, in addition to Collinsella aerofaciens. For example, it may comprise some strains of the following genus : Bifidobacterium, Lactobacillus, Lactococcus, Streptococcus, Enterococcus or of the following species : Lactobacillus acidophilus, Lactobacillus plantarum, Lactobacillus casei subsp. casei, Lactobacillus casei subsp. rhamnosus, Lactobacillus lactis, Lactobacillus helveticus, Lactobacillus casei, Lactobacillus paracasei, Lactobacillus cremoris, Lactobacillus rhamnosus, Lactobacillus delbrueckii subsp. bulgaricus, Lactobacillus delbrueckii subsp. lactis, Lactobacillus delbrueckii subsp. delbrueckii, Lactobacillus delbrueckii subsp. bulgaricus, Lactobacillus reuteri, Lactobacillus amylovorus, Lactobacillus johnsonii, Lactobacillus fermentum, Lactobacillus brevis, Streptococcus thermophilus, Lactococcus lactis subsp. lactis, Lactococcus lactis subsp. cremoris, Bifidobacterium longum, Bifidobacterium breve, Bifidobacterium bifidum, Bifidobacterium infantis, Bifidobacterium lactis, Bifidobacterium animalis, Bifidobacterium animalis subsp. lactis, Bifidobacterium infantis, and Bifidobacterium adolescentis.

Another object of the present invention is the use of a compound able to increase the intestinal population and/or the metabolic activity of Collinsella aerofaciens for reducing intestinal bloating and/or feeling of bloating. By way of example of compounds able to increase the intestinal population and/or the metabolic activity of Collinsella aerofaciens, one can mention fructo- and galacto-oligosaccharides, as reported by Tannock et al. (Appl Enviro. Microbiol., 70, 2129-2136, 2004).

The present invention will be understood more clearly with the aid of the additional description which follows, which refers to a non limiting example illustrating the relation between the intestinal population of Collinsella aerofaciens and the reduction of bloating and/or of the feeling of bloating in subjects suffering from IBS-C.

### EXAMPLE

### Correlation Between Collinsella Aerofaciens Population and Bloating Score in IBS-C Patients

A study on the effects on IBS symptomatology of 4 weeks consumption of a fermented milk product containing Bifidobacterium lactis DN-173 010 (test group) vs. a milk-based non-fermented dairy product without probiotics (control group) was conduced in 34 female patients (17 in each group) with IBS-C. The protocol and the results of this study are described in Agrawal et al., (Aliment Pharmacol Ther, 29, 104-114, 2008). One of the symptoms analysed in this study was abdominal bloating, evaluated by the subjects on a 0-5 scale. This symptom was found to be alleviated in the test group.

Along with the assessment of IBS symptomatology, stool samples were collected at baseline (Day 0) and following 4-week consumption period, for analysis of the fecal microbial population. The samples were stored in RNAlater® stabilization reagent until RNA isolation.

The Collinsella aerofaciens population in fecal samples was quantified by reverse transcription-quantitative PCR (RT-qPCR), according to the protocol described by Matsuda et al. (Appl. Environ. Microbiol.; 75, 1961-1969, 2009), using the following primers, derived from Collinsella aerofaciens ATCC 25986T;

| (SEQ ID NO: 1) | |
|---|---|
| Forward primer: | CCCGACGGGAGGGGA |
| (SEQ ID NO: 2) | |
| Reverse primer: | CTTCTGCAGGTACAGTCTTGA |

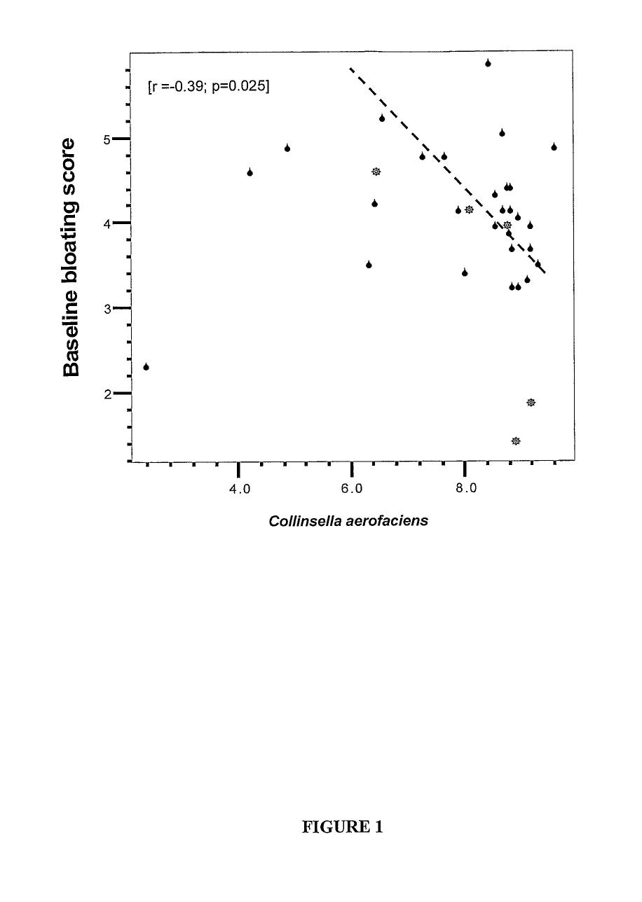

FIG. 1 represents the graph illustrating the relation at base line between the population of Collinsella aerofaciens (log₁₀cells/g) and the severity of the bloating symptoms (0-5 scale). This graph shows that the levels of Collinsella aerofaciens are inversely correlated with bloating symptoms severity (r=-0,39, p<0,01).

After the 4-week consumption period, the test group had significantly higher mean values of Collinsella aerofaciens compared with the control group (8.41 vs 7.55 log₁₀cells/g of feces, p=0.028, ANCOVA adjusted to baseline).

1. A composition comprising an effective amount of Collinsella aerofaciens for reducing intestinal bloating in a subject.

2. The composition of claim 1, wherein the subject is suffering from irritable bowel syndrome (IBS).

3. The composition of claim 1, wherein the Collinsella aerofaciens is in a concentration range of about 10³ CFU/ml to about 10¹¹ CFU/ml.

4. A method of reducing intestinal bloating comprising administering to a subject in need thereof, a therapeutically effective amount of the composition of claim 1.

5. The method of claim 4, wherein the subject is suffering from IBS.

6. The method of claim 5, wherein the IBS is IBS with constipation (IBS-C).


SEQUENCE LISTING (2 sequences)

SEQ ID NO: 1 - DNA, 15 bp, Collinsella aerofaciens
cccgacgggagggga

SEQ ID NO: 2 - DNA, 21 bp, Collinsella aerofaciens
cttctgcaggtacagtcttga